(19) United States
(12) Patent Application Publication
(10) Pub. No.: US 2024/0100178 A1
(43) Pub. Date: Mar. 28, 2024

(54) EGFR TARGETING PEPTIDE CONJUGATE

(71) Applicant: Chapman University, Orange, CA (US)

(72) Inventors: Kamaljit Kaur, Orange, CA (US); Phi-Phung Than, Orange, CA (US)

(21) Appl. No.: 18/452,405

(22) Filed: Aug. 18, 2023

Related U.S. Application Data
(60) Provisional application No. 63/399,010, filed on Aug. 18, 2022.

(51) Int. Cl. A61K 47/68; A61P 35/00; C07K 7/08

(52) U.S. Cl. CPC A61K 47/6809; A61P 35/00; C07K 7/08

(57) ABSTRACT

Disclosed herein are peptide-pharmaceutical agent conjugates and methods of treating cancer with the conjugates.

### CROSS-REFERENCE TO RELATED APPLICATIONS

The present application claims the benefit of U.S. Provisional Patent Application 63/399,010 filed Aug. 18, 2022, the entire contents of which is incorporated by reference herein.

### STATEMENT REGARDING FEDERALLY-SPONSORED RESEARCH

This invention was made with government support under Grant No. R15CA208656 awarded by the National Cancer Institute of the National Institutes of Health. The government has certain rights in the invention.

### SEQUENCE LISTING

This application contains a sequence listing having the filename 1959206-00036_Sequence Listing.xml, which is 8 KB in size, and was created on Aug. 18, 2023. The entire content of this sequence listing is incorporated herein by reference.

### FIELD

The present disclosure is directed to peptide-drug conjugates for treating cancer.

### BACKGROUND

Some cancer chemotherapeutic agents have low blood-brain barrier (BBB) permeability due to the multidrug resistance efflux pump P-glycoprotein (P-gp) expression in brain tumor cells. The U.S. Food and Drug Administration (FDA) has approved several antibody-drug conjugates (ADCs) over the last decade where the antibody targets cell-surface receptors (such as Trop2, HER2, CD22, CD30, or CD 33) on cancer cells for specific uptake and toxicity to cancer cells. Peptide-drug conjugates are challenging to engineer compared to ADCs; however, they have several advantages over ADCs, such as low manufacturing costs, easy/precise synthesis (as opposed to ADCs that give heterogeneous mixtures with batch-to-batch variations and complex pharmacokinetics), better ability to cross the BBB as several cancer types tend to metastasize to the brain, and less risk of immunogenicity.

### SUMMARY

Disclosed herein are drug delivery systems comprising a peptide targeting agent and a pharmaceutical agent for the treatment of cancer.

Disclosed herein is a peptide-drug conjugate, wherein the peptide comprises one of SEQ ID NOs:1-5. In some embodiments, the drug is a chemotherapeutic agent or a radioactive agent.

In some embodiments, the chemotherapeutic agent is selected from Itretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, nab-paclitaxel, paclitaxel, vinblastine, vincristine, vinorelbine, all-trans-retinoic acid, arsenic trioxide, asparaginase, eribulin, hydroxyurea, ixabepilone, mitotane, omacetaxine, pegaspargase, procarbazine, romidepsin, and vorinostat. In some embodiments, the chemotherapeutic agent comprises doxorubicin.

In some embodiments, the peptide comprises SEQ ID NO:1. In some embodiments, the peptide comprises SEQ ID NO:2. In some embodiments, the peptide comprises SEQ ID NO:3. In some embodiments, the peptide comprises SEQ ID NO:4. In some embodiments, the peptide comprises SEQ ID NO:5.

In some embodiments, the peptide-drug conjugate further comprises a linker linking the peptide to the pharmaceutical agent. In some embodiments, the linker comprises a thioester, an amide, a carbamate, an ester, or a carbonate.

In some embodiments, the peptide-drug conjugate comprises the peptide of SEQ ID NO:5 conjugated to doxorubicin.

Also disclosed herein are pharmaceutical compositions comprising the peptide-drug conjugate disclosed herein.

Also disclosed herein are methods of treating cancer comprising administering to a cancer patient in need thereof a peptide-drug conjugate disclosed herein.

In some embodiments, the cancer is selected from acute lymphoblastic leukemia, acute myeloid leukemia, adrenocortical carcinoma, anal cancer, astrocytoma, bile duct cancer, bladder cancer, bone cancer, brain tumor, breast cancer, carcinoid tumor, carcinoma, cervical cancer, chronic lymphocytic leukemia, chronic myelogenous leukemia, myeloproliferative disorders, colorectal cancer, cutaneous T-cell lymphoma, endometrial cancer, ependymoma, esophageal cancer, Ewing's family of tumors, germ cell tumor, retinoblastoma; gallbladder cancer, gastric cancer, glioma, hairy cell leukemia, head and neck cancer; hepatocellular cancer, Hodgkin's lymphoma, islet cell carcinoma, Kaposi's sarcoma, kidney cancer, laryngeal cancer, lip and oral cavity cancer, liver cancer, lung cancer, non-small cell lung cancer, small cell lung cancer, lymphoblastic leukemia, lymphocytic leukemia, lymphoma, non-Hodgkin's lymphoma, Waldenstrom's macroglobulinemia, mesothelioma, malignant thymoma, medulloblastoma, melanoma, multiple endocrine neoplasia syndrome, multiple myeloma/plasma cell neoplasm, mycosis fungoides, myelodysplastic syndromes, myelogenous leukemia, myeloid leukemia, nasopharyngeal cancer, neuroblastoma, oral cancer, oropharyngeal cancer, osteosarcoma/malignant fibrous histiocytoma of bone, ovarian cancer, pancreatic cancer, parathyroid cancer, penile cancer, pheochromocytoma, pituitary tumor, prostate cancer, rectal cancer, renal cell cancer, retinoblastoma, rhabdomyosarcoma, salivary gland cancer, sarcoma, Sezary syndrome, skin cancer, soft tissue sarcoma, squamous neck cancer, testicular cancer, thymoma, thyroid cancer, trophoblastic tumor, urethral cancer, uterine sarcoma, vaginal cancer, vulvar cancer, and Wilms' tumor.

In some embodiments, the cancer is breast cancer. In some embodiments, the breast cancer is triple negative breast cancer.

### BRIEF DESCRIPTION OF THE DRAWINGS

FIG. 1 depicts the structure of the novel peptide-doxorubicin conjugate (PDoxC) targeting EGFR. Peptide, linker, and doxorubicin are shown in black, blue, and red, respectively.

FIG. 2 depicts the stability of peptide GE11 in the presence of human serum at 37° C. by RP-HPLC chromatograms of peptide GE11 alone and peptide after incubation with human serum at different time intervals (0, 1, 3, and 7 h). Peptide was incubated with human serum at 37° C., and aliquots were removed at different time intervals for HPLC analysis.

FIG. 3 depicts the stability of the PDoxC in the presence of human or mouse serum at 37° C. The PDoxC was incubated with human or mouse serum and aliquots were removed at different time intervals for HPLC analysis. The AUC for PDoxC peak was used to calculate the percent intact at different time points.

FIG. 4A-C depicts the cytotoxicity of PDoxC, obtained by MTT assay, for three cell lines (MDA-MB-231 [FIG. 4A], MDA-MB-468 [FIG. 4B], and MCF-10A [FIG. 4C]). Cell viability is plotted against drug concentration (PDoxC or Dox) to obtain IC₅₀ values. Each experimental point was done in duplicates or triplicates and the experiment was repeated once.

### DETAILED DESCRIPTION

Provided herein are drug delivery systems comprising a peptide targeting agent and a pharmaceutical agent for delivery of chemotherapeutic agents. Also provided are methods of treating cancer comprising administration of the drug delivery system disclosed herein.

The inherent resistance in cells that have never been exposed to anticancer drugs and the development of resistance after the initial response is one of the major limitations of cancer chemotherapy treatment. Several mechanisms of inherent and acquired multi-drug resistance have been studied, including alteration of the target protein and drug metabolism, decreased membrane permeability, and/or efflux pumping.

One of these chemotherapeutic agents, doxorubicin (Dox), is used for treatment of breast cancer, leukemia, and lymphoma as an effective chemotherapeutic agent. However, Dox use is restricted due to inherent and acquired resistance, an 8-fold increase in the risk of potentially fatal cardiotoxicity, and development of resistance associated with it. Doxorubicin is a well-known, widely used anthracycline anticancer agent and has been approved by FDA for the treatment of leukemias, sarcomas, and lymphoma, as well as breast, gastric, ovarian, lung, and thyroid cancer. Dox acts through the inhibition of topoisomerase II (TOPO II)-DNA complex. DNA damage occurs by intercalating of Dox with the DNA double helix.

Moreover, the clinical application of Dox has demonstrated undesirable pharmacokinetic properties, such as low bioavailability, rapid distribution, and excretion of the drug, due to the hydrophilic nature and a high volume of distribution. Thus, a higher dose of Dox is required in cancer chemotherapy to achieve an adequate therapeutic effect. However, a higher cumulative dose leads to dose-dependent side effects, such as cardiotoxicity, nephrotoxicity, and extravasation.

Intracellular chemotherapeutic agent accumulation is dependent on many parameters, such as cellular uptake, retention, re-localization, and efflux from the cell. Among these factors, uptake of some agents is affected by the efflux mechanism in a number of cancer cells such as ovarian carcinoma cells which leads to the decreased levels of intracellular agent. The overexpression of energy-dependent efflux pump integral membrane proteins such as P-glycoprotein (P-gp) removes drugs and thus reduces intracellular anticancer drug concentrations.

The biological efficacy and toxicity of an anticancer drug can be modified by using drug delivery systems and altering the physicochemical properties, such as lipophilicity, cellular uptake, and prolonging activity through chemical conjugation with various chemical moieties. One of the main applications of drug delivery systems is avoiding the P-gp and multidrug resistance proteins (MRPs) involved in drug efflux to overcome the resistance problem and P-gp-mediated drug efflux.

Chemical conjugation with a parent drug has been widely used as one of the drug delivery systems, which is referred to as a prodrug strategy. Several methods have been used to improve chemotherapeutic agent delivery, including using gold nanoparticles, gold nanospheres, liposomes, peptides, and dendrimers. Several delivery systems of chemotherapeutic agents have been explored, including metal nanoparticles, carbon nanotubes, dendrimers, liposomes, fullerenes, cyclic peptides, and other covalent/non-covalent systems).

The epidermal growth factor receptor (EGFR) is overexpressed in several cancers, including triple-negative breast cancer (TNBC). It has been shown that several mechanisms lead to EGFR overexpression in TNBC, including EGFR gene amplification. The overexpression of the target cell-surface protein makes it ideal for developing targeted drug conjugates, such as peptide-drug conjugates (PDCs) or antibody-drug conjugates (ADCs), which do not rely on downstream signaling and depend on target expression. EGFR is a glycoprotein consisting of an extracellular N-terminal binding domain, a hydrophobic transmembrane (TM) region, and an intracellular C-terminal tyrosine kinase (TK) domain. EGFR belongs to a family of receptors consisting of 4 different types of proteins (EGFR or ErbB1, ErbB2, ErbB3, and ErbB4) that are activated following binding to their ligands which include EGF. Ligand binding activates various signaling pathways that promote cell processes such as, proliferation, angiogenesis, tumor survival, and metastasis. EGFR activation normally leads to cellular growth and its signaling can provide substantial advantages in tumor cells' survival, therefore, specific EGFR inhibition is one of the key strategies in cancer therapy. In addition, ligand binding to EGFR induces EGFR endocytosis and can be utilized for targeted receptor-mediated uptake of drug conjugates such as peptide-drug conjugates. EGFR is often overexpressed and/or mutated in a number of tumors of epithelial origin including TNBC. Specific mutations in the extracellular or intracellular domains have been reported for EGFR in glioblastoma, lung, and colorectal cancers. Numerous EGFR blockers have been investigated which include anti-EGFR monoclonal antibodies, such as cetuximab. However, clinical trials with monoclonal antibodies and/or tyrosine kinase inhibitors against EGFR failed to demonstrate significant activity in TNBC patients warranting other strategies for treatment of TNBC. In this regard, research efforts are being directed toward EGFR-binding ligands in cancer cells for conjugation to drugs or nanosystems to achieve tumor specific drug delivery and internalization e.g., PDCs or ADCs. An ADC, ABT-414, which selectively kills cancer cells overexpressing wild-type or mutant EGFR, has entered phase I clinical trials. Overall the studies and data presented herein support that EGFR overexpressed in TNBC cells can be utilized for specific and targeted uptake of PDCs for improved efficacy of chemotherapy for TNBC treatment.

Peptide Targeting Pharmaceutical Agent Conjugates

Disclosed herein are drug delivery systems comprising a peptide targeting agent and a pharmaceutical agent. In some embodiments, the drug delivery system comprises a peptide-drug conjugate.

In some embodiments, the peptide comprises (X¹)ₙYHWYGYTPX²X³VI (SEQ ID NO:1). In some embodiments X¹ is C wherein n is 0 or 1. In some embodiments X² is Q or E. In some embodiments X³ is N, K, or R. In some embodiments, the peptide comprises YHWYGYTPQNVI (SEQ ID NO:2), YHWYGYTPENVI (SEQ ID NO:3), YHWYGYTPQKVI (SEQ ID NO:4), or YHWYGYTPERVI (SEQ ID NO:5).

In some embodiments, the pharmaceutical agent is a chemotherapeutic agent. In some embodiments, the chemotherapeutic agent is selected from altretamine, bendamustine, busulfan, carboplatin, carmustine, chlorambucil, cisplatin, cyclophosphamide, dacarbazine, doxorubicin, ifosfamide, lomustine, mechlorethamine, melphalan, oxaliplatin, temozolomide, thiotepa, trabectedin, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, nab-paclitaxel, paclitaxel, vinblastine, vincristine, vinorelbine, all-trans-retinoic acid, arsenic trioxide, asparaginase, eribulin, hydroxyurea, ixabepilone, mitotane, omacetaxine, pegaspargase, procarbazine, romidepsin, and vorinostat, and combinations thereof. In some embodiments, the chemotherapeutic agent is doxorubicin.

In some embodiments, the pharmaceutical agent is a radioactive agent. Non-limiting examples of radionuclides suitable for use in the PDCs disclosed herein include ¹³¹I, ²²³Ra, ⁸⁹Sr, ¹⁵³Sm, ³²P, ⁹⁰Y, ¹⁷⁷Lu, ²¹³Bi, ⁹⁹Tc, ¹¹¹In, ²¹¹At, ¹⁶⁶Ho, ¹⁸⁶Re, ¹⁸⁸Re, ⁶⁷Cu, ¹⁴⁹Promethium, ¹⁹⁹Au, ¹⁰⁵Rh, ⁷⁷Br, ¹²³I, and ¹²⁵I.

In some embodiments, the peptide-drug conjugate comprises YHWYGYTPQNVI (SEQ ID NO:2) conjugated to a chemotherapeutic agent, such as doxorubicin. In some embodiments, the peptide-drug conjugate comprises YHWYGYTPENVI (SEQ ID NO:3) conjugated to a chemotherapeutic agent, such as doxorubicin. In some embodiments, the peptide-drug conjugate comprises YHWYGYTPQKVI (SEQ ID NO:4) conjugated to a chemotherapeutic agent, such as doxorubicin In some embodiments, the peptide-drug conjugate comprises YHWYGYTPERVI (SEQ ID NO:5) conjugated to a chemotherapeutic agent, such as doxorubicin.

Conjugation to a chemotherapeutic agent, such as doxorubicin, to a peptide disclosed herein is performed by conjugation methods known by persons of ordinary skill in the art such as enzyme-cleavable linker chemistries, acid-cleavable linker chemistries, reducible disulfide linker chemistries, and non-cleavable linker chemistries. In some embodiments, the enzyme-cleavable linker chemistry includes, but is not limited to, ester or amide, carbamate, amide+thioether, and ester/triazole. In some embodiments, the acid-cleavable linker chemistry includes, but is not limited to, a hydrazine linker. In some embodiments, the non-cleavable linker chemistry includes, but is not limited to, succinimidyl thioether, oxime, and triazole. Exemplary bonds for peptide-drug conjugates are found in Alas et al. (J Med Chem 64:216-232, 2021). In some embodiments, the conjugate is through an amide, ester, carbamate, carbonate, or thioester linkage.

In some embodiments, a dipeptide or tripeptide linker is used which facilitates release of active drug from the peptide-drug conjugate. A non-limiting exemplary dipeptide linker is Val-Cit. A non-limiting exemplary tripeptide linker is Asn-Ala-Ala.

Treatment

Examples of cancers which can be treated by the disclosed methods include acute lymphoblastic leukemia, acute myeloid leukemia, adrenocortical carcinoma, anal cancer, astrocytoma, bile duct cancer, bladder cancer, bone cancer, brain tumor, breast cancer, carcinoid tumor, carcinoma, cervical cancer, chronic lymphocytic leukemia, chronic myelogenous leukemia, myeloproliferative disorders, colorectal cancer, cutaneous T-cell lymphoma, endometrial cancer, ependymoma, esophageal cancer, Ewing's family of tumors, germ cell tumor, retinoblastoma; gallbladder cancer, gastric cancer, glioma, hairy cell leukemia, head and neck cancer; hepatocellular cancer, Hodgkin's lymphoma, islet cell carcinoma, Kaposi's sarcoma, kidney cancer, laryngeal cancer, lip and oral cavity cancer, liver cancer, lung cancer, non-small cell lung cancer, small cell lung cancer, lymphoblastic leukemia, lymphocytic leukemia, lymphoma, non-Hodgkin's lymphoma, Waldenstrom's macroglobulinemia, mesothelioma, malignant thymoma, medulloblastoma, melanoma, multiple endocrine neoplasia syndrome, multiple myeloma/plasma cell neoplasm, mycosis fungoides, myelodysplastic syndromes, myelogenous leukemia, myeloid leukemia, nasopharyngeal cancer, neuroblastoma, oral cancer, oropharyngeal cancer, osteosarcoma/malignant fibrous histiocytoma of bone, ovarian cancer, pancreatic cancer, parathyroid cancer, penile cancer, pheochromocytoma, pituitary tumor, prostate cancer, rectal cancer, renal cell cancer, retinoblastoma, rhabdomyosarcoma, salivary gland cancer, sarcoma, Sezary syndrome, skin cancer, soft tissue sarcoma, squamous neck cancer, testicular cancer, thymoma, thyroid cancer, trophoblastic tumor, urethral cancer, uterine sarcoma, vaginal cancer, vulvar cancer, and Wilms' tumor. In certain embodiments, the cancer is breast cancer. In some embodiments, the cancer is a triple negative breast cancer. Some embodiments specifically include one or more of these cancers. Other embodiments specifically exclude one or more of these cancers.

As used herein, the terms “treatment,” “treating,” and the like refer to obtaining a desired pharmacologic and/or physiologic effect. This may be observed directly as a slowing of tumor growth, stabilization of disease, or a partial or complete response (that is, tumor regression or elimination of tumors), or extended overall or disease-free survival. Treatment may also be observed as an amelioration or reduction of symptoms related to the underlying cancer. However, as cancer treatment, the disclosed embodiments' aim and mechanism is directed to inhibiting, stabilizing, or reducing tumor growth (including metastases), or partially or completely eliminating tumors, or extending overall or disease-free survival; effects on other cancer symptoms are secondary. Direct treatment of such other symptoms (for example, pain, nausea, loss of appetite, etc.) is not within the scope of treating cancer as used herein. That is, treating a symptom, for example, cachexia in a cancer patient is not treating cancer. However, an agent that treats cancer (e.g., has an impact on the growth and/or spread of cancer) may also ameliorate a symptom, such as cachexia, either indirectly, through its effect on the cancer, or directly, through a pleiotropic effect. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of a composition disclosed herein to elicit a desired response in the individual.

However, the dose administered to a mammal, particularly a human, in the context of the present methods, should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like that are then acted upon by any other person including other healthcare professionals or the patient his/herself. In some embodiments, treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like.

To benefit from the combined effect of peptide-drug conjugates disclosed herein, embodiments include methods of treatment comprising or consisting of administering a peptide-drug conjugates to a patient having cancer.

In various embodiments the herein disclosed treatments may be applied as a primary therapy, as a debulking therapy prior to surgical removal of tumor, or as an adjuvant therapy subsequent to any mode of primary therapy (especially surgery) to address residual disease and/or lower the risk of recurrent cancer.

In some embodiments the patient having cancer has not been previously treated with the conjugated pharmaceutical agent. In some embodiments the patient has been previously treated with the conjugated pharmaceutical agent and has achieved stable disease or a partial response (in some embodiments, as defined by RECIST or iRECIST criteria)—that is, the cancer is sensitive to the conjugated pharmaceutical agent.

Therapeutic efficacy can be monitored by periodic assessment of treated patients. For repeated administrations over several days or longer, the treatment can be repeated until a desired suppression of disease or disease symptoms occurs. However, other dosage regimens may be useful and are within the scope of the present disclosure.

The effectiveness of cancer therapy is typically measured in terms of “response.” The techniques to monitor responses can be similar to the tests used to diagnose cancer such as, but not limited to:
A lump or tumor involving some lymph nodes can be felt and measured externally by physical examination.
Some internal cancer tumors will show up on an x-ray or CT scan and can be measured with a ruler.
Blood tests, including those that measure organ function can be performed.
A tumor marker test can be done for certain cancers.

Regardless of the test used, whether blood test, cell count, or tumor marker test, it is repeated at specific intervals so that the results can be compared to earlier tests of the same type.

Response to cancer treatment is defined several ways:
Complete response—all of the cancer or tumor disappears; there is no evidence of disease. Expression level of tumor marker (if applicable) may fall within the normal range.
Partial response—the cancer has shrunk by a percentage but disease remains. Levels of a tumor marker (if applicable) may have fallen (or increased, based on the tumor marker, as an indication of decreased tumor burden) but evidence of disease remains.
Stable disease—the cancer has neither grown nor shrunk; the amount of disease has not changed. A tumor marker (if applicable) has not changed significantly.
Disease progression—the cancer has grown; there is more disease now than before treatment. A tumor marker test (if applicable) shows that a tumor marker has risen.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization or partial response that resumed tumor growth is not detectable).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis. There is also a variation of the RECIST method for immunotherapies (iRECIST) which takes into account distinctive behaviors linked to these types of therapeutics, such as delayed responses after pseudoprogression. Both the RECIST 1.1 guidelines and the iRecist guidelines are incorporated by reference herein in their entirety.

In some embodiments of the herein disclosed methods, the tumor burden of a treated patient is reduced by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 1-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 5-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 10-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In yet other embodiments, the subject has a sustained remission of at least 6 months, at least 7 months, at least 8 months, at least 9 months, at least 10 months, at least 11 months, at least 12 months, at least 14 months, at least 16 months, at least 18 months, at least 20 months, at least 22 months, at least 24 months, at least 27 months, at least 30 months, at least 33 months, at least 36 months, at least 42 months, at least 48 months, at least 54 months, or at least 60 months or more.

While a cancer treatment may reduce or treat associated symptoms, treating symptoms associated with cancer, is not treating cancer if there is no expectation that tumor will be reduced or eliminated or their growth or spread will be inhibited.

Toxicities and adverse events are sometimes graded according to a 5 point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death.

### EXAMPLES

### Example 1

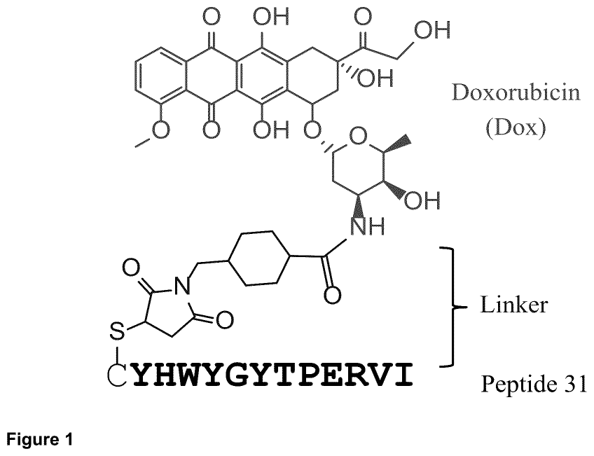

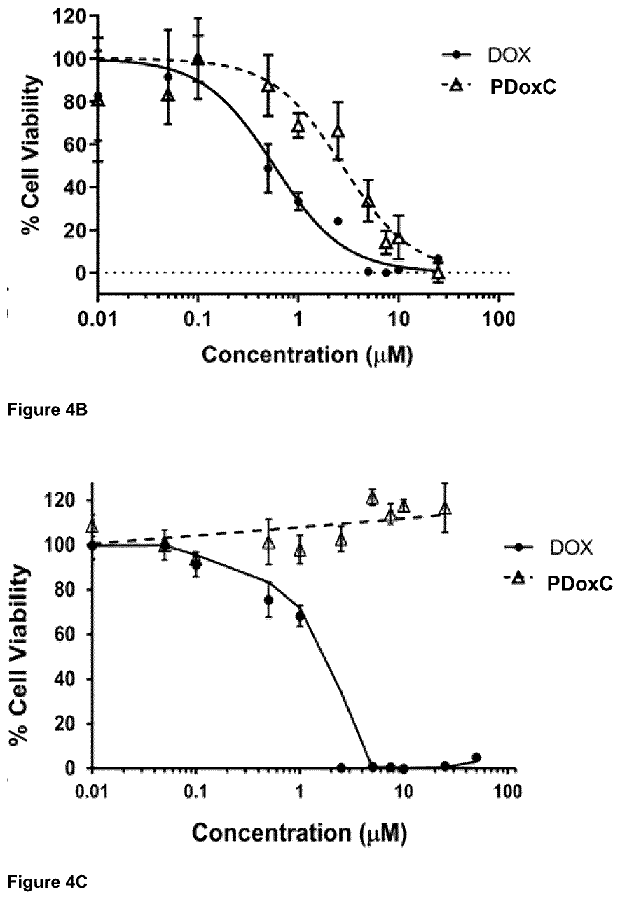

Disclosed herein is the discovery of a novel EGFR binding peptide 31 (Table 1) for specific binding (uptake) by TNBC cells. The peptide was conjugated to the chemotherapeutic agent doxorubicin (DOX) via a thioether linker (MCC) to obtain a novel peptide-doxorubicin conjugate (PDoxC, FIG. 1). The PDoxC shows specific toxicity to TNBC cells (MDA-MB-231 and MDA-MB-468) and no toxicity to normal breast tissue-derived MCF-10A cells (FIG. 4).

TABLE 1

| SEQ | | |
|---|---|---|
| ID | Peptide | |
| NO: | designation | Sequence |
| 2 | GE11 | YHWYGYTPQNVI |
| 3 | 22 | YHWYGYTPENVI |
| 4 | 27 | YHWYGYTPQKVI |
| 5 | 31 | YHWYGYTPERVI |
| 6 | 23 | YHWYGYTPQDVI |
| 7 | 26 | YHWYGYTPKNVI |

The discovery of EGFR targeting peptide 31 started with peptide GE11 (FIG. 1). GE11 (YHWYGYTPQNVI) is a 12-mer peptide ligand specific for EGFR, which was originally identified by screening a phage display library against purified hEGFR protein. The GE11 peptide shows high binding affinity to EGFR (Kd=22 nM), without triggering dimerization, and mitosis like its endogenous ligand, EGF. The peptide is internalized by cells overexpressing EGFR and when delivered i.v. in mice it accumulates in EGFR overexpressing tumor xenografts. This peptide has been used to develop cancer targeted therapeutics, diagnostics, or both.

A library of peptides, using GE11 as the lead peptide, was screened to identify EGFR specific peptides (1^{st} generation TNBC targeting peptides) for specific uptake by TNBC cells. A library of 30 peptide sequences (in duplicates, total 60 peptides) was synthesized in an array format on functionalized cellulose membrane using SPOT synthesis. The peptide library was screened for identifying EGFR binding peptide in TNBC cells by incubating the library with TNBC cells (MDA-MB-468, 231, and 436) or normal breast (MCF-10A and 12A) cells. Four peptides (22, 23, 26, and 27) were identified with higher specific affinity for TNBC cells (Table 1).

Peptide 22 showed several-fold (47-123 fold) higher uptake by EGFR expressing TNBC cells (MDA-MB-231 and MDA-MB-468) compared to the lead GE11 and minimal uptake by the noncancerous mammary epithelial cells (MCF-10A and MCF12A). The specific binding and uptake of peptide 22 (FITC labeled peptide) by EGFR in TNBC cells was confirmed by competition assay using flow cytometry. The uptake of peptide 22 was considerably reduced when the cells were incubated with peptide 22 in the presence of excess EGF protein, as evidenced by the reduction in the FITC-positive MDA-MB-231 cells from 99% to 2%.

### Results

From the first generation peptides, here we have designed a second generation EGFR binding peptide (peptide 31, Table 1) for targeting TNBC cells. Peptide 31 has two substitutions (9E and 10R) derived from peptides 22 and 27, compared to peptide GE11. Also, Arg (R) is substituted at position 10 instead of K (in peptide 27) due to the presence of ER in the EGR protein sequence. Peptide 31 has high sequence homology with a portion of EGF, an endogenous protein (53 amino acid residues) that binds and activates EGFR.

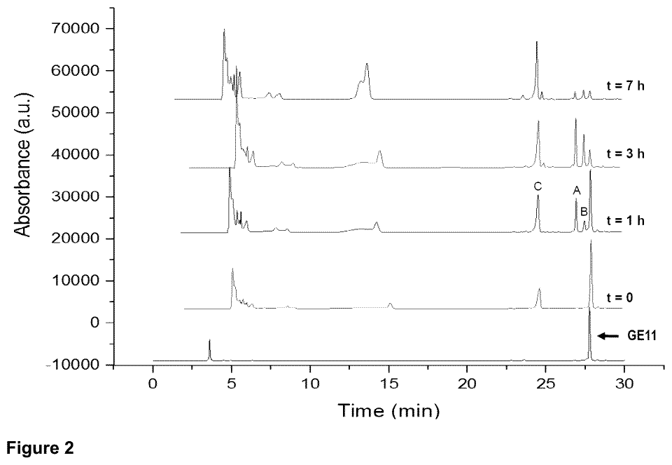

Peptides show low proteolytic stability when administered systemically and must be stabilized for clinical application. We studied the stability of peptide GE11 by incubating the peptide with human serum at 37° C. It was found that the peptide gets cleaved from the N-terminus when incubated with human serum (FIG. 2 and Tables 2 and 3). The three N-terminal amino acids (1Y, 2H, 3W) were cleaved sequentially by proteolysis, giving three fragments (A, B, and C, Table 2)) that appear in the HPLC chromatograms. After incubation with human serum for 7 hours, most of the peptide GE11 was degraded (2% intact) (Table 3). Therefore, blocking the N-terminus of the peptide with a chemotherapeutic drug yields a stable conjugate.

TABLE 2

| SEQ | | |
|---|---|---|
| ID | Peptide | |
| NO: | designation | Sequence |
| 2 | GE11 | YHWYGYTPQNVI |
| 6 | Fragment A | HWYGYTPQNVI |
| 7 | Fragment B | WYGYTPQNVI |
| 8 | Fragment C | YGYTPQNVI |

TABLE 3

| Time (h) | % Intact GE11 |
|---|---|
| 0 | 100 |
| 1 | 49 |
| 3 | 18 |
| 7 | 2 |

We synthesized a conjugate of peptide 31 with doxorubicin (DOX) where the N-terminus of the peptide was attached to DOX via a succinimidyl thioether linkage (FIG. 1). The new PDC was synthesized as previously described (Ziaei E. et al. Bioconj Chem 30:3098-3106 doi:10.1021/acs.bioconjchem.9b00755, 2019). The peptide 31, and the PDoxC were characterized using RP-HPLC and MALDI-TOF mass spectrometry. The PDoxC was purified using RP-HPLC to give >95% pure product.

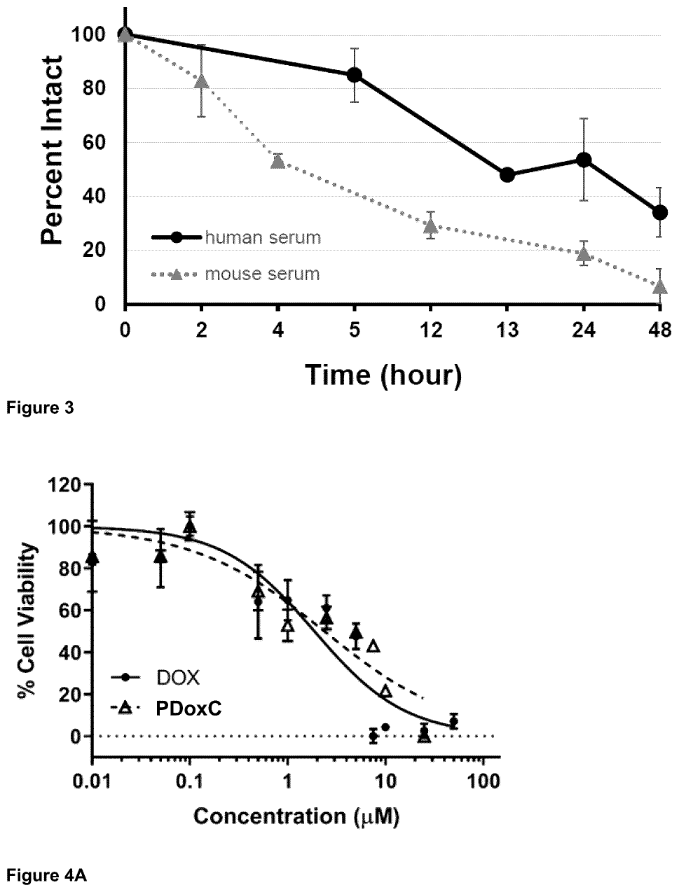

The stability of the peptide 31 PDoxC was evaluated by incubating it with human or mouse serum at 37° C. (FIG. 3). Aliquots were removed at regular intervals to analyze the PDoxC using RP-HPLC. The HPLC peak (area under the curve) for the peptide 31 PDoxC decreased overtime with a half-life of 12.7 and 4 hours for human and mouse serum, respectively. The results suggest that the peptide 31 PDoxC is stable for in vivo applications, as the PDoxC is expected to reach tumor site within an hour or less.

Next, the cytotoxicity of the peptide 31 PDoxC was compared to the DOX (free drug) using MTS assay (FIG. 4A-C). Three cell lines were used, two TNBC cell lines (MDA-MB-231 and MDA-MB-468) and one non-cancerous mammary epithelial MCF-10A cell line. The peptide 31 PDoxC and DOX showed high toxicity to the TNBC cells. The IC₅₀ values of DOX for MDA-231 and MDA-468 cells were 1.87 μM and 0.57 μM, respectively (Table 4). Similarly, IC₅₀ values of the PDoxC were in low micromolar range for the TNBC cells with values of 2.37 μM and 2.75 μM, respectively. Dox was highly toxic to normal mammary MCF-10A cells with IC50 value of 1.41 μM while the PDoxC showed no toxicity up to the highest tested concentration of 25 μM. This peptide 31 PDoxC is very promising as this level of selectivity has never been observed with previously reported peptide-drug conjugates.

TABLE 4

| | IC50 ± SD (μM) | | |
|---|---|---|---|
| Treatment | MDA-MB-231 | MDA-MB-468 | MCF-10A |
| Free Dox | 1.87 ± 0.36 | 0.57 ± 0.09 | 1.41 ± 0.07 |
| PDoxC | 2.37 ± 0.55 | 2.75 ± 0.57 | no toxicity |

Based on previous in vivo studies (using the mouse model) to show the efficacy of peptide-drug conjugates (Saghaeidehkordi, A., et al. Pharmaceutics 13:661, doi:10.3390/pharmaceutics13050661, 2021 and Ziaei, E., et al. Molecular Pharmaceutics 20:3570, doi:10.1021/acs.molpharmaceut.3c00189, 2023), the novel PDoxC is highly efficacious. The peptide can be conjugated to other chemotherapeutic drugs which are highly cytotoxic and therefore cannot be used alone. Peptide 31 has been developed for targeting TNBC and the peptide could be used for targeting other EGFR-expressing cancers. Finally, the peptide can be used for targeting chemotherapy, radiotherapy, or imaging agents.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A peptide-drug conjugate, wherein the peptide comprises one of SEQ ID NOs:1-5.

2. The peptide-drug conjugate of claim 1, wherein the drug is a chemotherapeutic agent or a radioactive agent.

3. The peptide-drug conjugate of claim 2, wherein the chemotherapeutic agent is selected from Itretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, streptozocin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, epirubicin, idarubicin, valrubicin, bleomycin, dactinomycin, mitomycin-c, mitoxantrone, irinotecan, topotecan, etoposide, mitoxantrone, teniposide, cabazitaxel, docetaxel, nab-paclitaxel, paclitaxel, vinblastine, vincristine, vinorelbine, all-trans-retinoic acid, arsenic trioxide, asparaginase, eribulin, hydroxyurea, ixabepilone, mitotane, omacetaxine, pegaspargase, procarbazine, romidepsin, and vorinostat.

4. The peptide-drug conjugate of claim 3, wherein the chemotherapeutic agent comprises doxorubicin.

5. The peptide-drug conjugate of claim 1, wherein the peptide comprises SEQ ID NO:1.

6. The peptide-drug conjugate of claim 1, wherein the peptide comprises SEQ ID NO:2.

7. The peptide-drug conjugate of claim 1, wherein the peptide comprises SEQ ID NO:3.

8. The peptide-drug conjugate of claim 1, wherein the peptide comprises SEQ ID NO:4.

9. The peptide-drug conjugate of claim 1, wherein the peptide comprises SEQ ID NO:5.

10. The peptide-drug conjugate of claim 1, further comprising a linker linking the peptide to the pharmaceutical agent.

11. The peptide-drug conjugate of claim 9, wherein the linker comprises a thioester, an amide, a carbamate, an ester, or a carbonate.

12. The peptide-drug conjugate of claim 8, wherein the peptide-drug conjugate comprises the peptide of SEQ ID NO:5 conjugated to doxorubicin.

13. A pharmaceutical composition comprising the peptide-drug conjugate of claim 1.

14. A method of treating cancer comprising administering to a cancer patient in need thereof the peptide-drug conjugate of claim 1.

15. The method of claim 14, wherein the cancer is selected from acute lymphoblastic leukemia, acute myeloid leukemia, adrenocortical carcinoma, anal cancer, astrocytoma, bile duct cancer, bladder cancer, bone cancer, brain tumor, breast cancer, carcinoid tumor, carcinoma, cervical cancer, chronic lymphocytic leukemia, chronic myelogenous leukemia, myeloproliferative disorders, colorectal cancer, cutaneous T-cell lymphoma, endometrial cancer, ependymoma, esophageal cancer, Ewing's family of tumors, germ cell tumor, retinoblastoma; gallbladder cancer, gastric cancer, glioma, hairy cell leukemia, head and neck cancer; hepatocellular cancer, Hodgkin's lymphoma, islet cell carcinoma, Kaposi's sarcoma, kidney cancer, laryngeal cancer, lip and oral cavity cancer, liver cancer, lung cancer, non-small cell lung cancer, small cell lung cancer, lymphoblastic leukemia, lymphocytic leukemia, lymphoma, non-Hodgkin's lymphoma, Waldenstrom's macroglobulinemia, mesothelioma, malignant thymoma, medulloblastoma, melanoma, multiple endocrine neoplasia syndrome, multiple myeloma/plasma cell neoplasm, mycosis fungoides, myelodysplastic syndromes, myelogenous leukemia, myeloid leukemia, nasopharyngeal cancer, neuroblastoma, oral cancer, oropharyngeal cancer, osteosarcoma/malignant fibrous histiocytoma of bone, ovarian cancer, pancreatic cancer, parathyroid cancer, penile cancer, pheochromocytoma, pituitary tumor, prostate cancer, rectal cancer, renal cell cancer, retinoblastoma, rhabdomyosarcoma, salivary gland cancer, sarcoma, Sezary syndrome, skin cancer, soft tissue sarcoma, squamous neck cancer, testicular cancer, thymoma, thyroid cancer, trophoblastic tumor, urethral cancer, uterine sarcoma, vaginal cancer, vulvar cancer, and Wilms' tumor:

16. The method of claim 15, wherein the cancer is breast cancer.

17. The method of claim 16, wherein the breast cancer is triple negative breast cancer.


SEQUENCE LISTING (7 sequences)

SEQ ID NO: 1 - protein, 13 aa, synthetic construct
  VARIANT 1: note = X=C
  VARIANT 9: note = X= Q or E
  VARIANT 10: note = X = N, K, or R
  REPEAT 1: note = 0 or 1
XYHWYGYTPXXVI

SEQ ID NO: 2 - protein, 12 aa, synthetic construct
YHWYGYTPQNVI

SEQ ID NO: 3 - protein, 12 aa, synthetic construct
YHWYGYTPENVI

SEQ ID NO: 4 - protein, 12 aa, synthetic construct
YHWYGYTPQKVI

SEQ ID NO: 5 - protein, 12 aa, synthetic construct
YHWYGYTPERVI

SEQ ID NO: 6 - protein, 12 aa, synthetic construct
YHWYGYTPQDVI

SEQ ID NO: 7 - protein, 12 aa, synthetic construct
YHWYGYTPKNVI